(19) United States
(12) Patent Application Publication
(10) Pub. No.: US 2020/0029571 A1
(43) Pub. Date: Jan. 30, 2020

(54) Bacterial strain bacillus amyloliquefaciens subsp plantarum bs89 as agent for increasing productivity of plants and protecting same from diseases

(71) Applicant: OBSCHESTVO S OGRANICHENNOI OTVETSTVENNBOSTJU "BISOLBI PLYUS", St.Petersburg (RU)

(72) Inventors: Vladimir Kuzmich CHEBOTAR, St.Petersburg (RU); Sergey Viktorovich EROFEEV, St.Petersburg (RU)

(21) Appl. No.: 15/756,871

(22) PCT Filed: Sep. 1, 2016
(86) PCT No.: PCT/RU16/00599

§ 371 Date: Mar. 1, 2018

(30) Foreign Application Priority Data
Sep. 2, 2015 (RU) 2015137387

(51) Int. Cl. A01N 63/02; C12R 1/07; C12N 1/20

(52) U.S. Cl. CPC A01N 63/02; C12N 1/20; C12R 1/07

(57) ABSTRACT

The invention relates to biotechnology and agriculture. Bacteria strain Bacillus amyloliquefaciens subsp. plantarum BS89 is an agent for increasing the productivity of plants and protecting them from diseases. The strain has been deposited in the Departmental Collection of the Federal State Funded Research Institution All-Russia Research Institute for Agricultural Microbiology under number RCAM 03458. The strain exhibits high fungicidal activity against phytopathogenic fungi and bactericidal activity against phytopathogenic bacteria, as well as high growth-promoting activity with respect to various crops, particularly wheat, barley, potatoes, cabbage, sugar beet, flax and sunflowers.

### CROSS-REFERENCE TO RELATED APPLICATIONS

This application is a U.S. national stage application of an international application PCT/RU2016/000599 filed on 1 Sep. 2016, published as WO/2017/039491, whose disclosure is incorporated herein in its entirety by reference, which international application claims priority of a Russian Federation patent application RU2015137387 filed on 2 Sep. 2015.

### FIELD OF THE INVENTION

The invention belongs to the sphere of biotechnology and agriculture and concerns a new strain of rhizosphere bacteria from the genus Bacillus as a means of increasing plant productivity and protecting plants from phytopathogenic microorganisms.

### BACKGROUND OF THE INVENTION

A highly efficient agricultural production is nowadays impossible without the application of fertilizers and plant protection products. So, owing to a broad use of mineral fertilizers (first of all, nitrogen fertilizers) the yield of the major agricultural crops in developed countries was increased more than 5-fold in the last fifty years. However, production and application of mineral nitrogen fertilizers is an energy-demanding process, consuming from 30 to 50% of all the energy used in agricultural production.

Numerous microbiological preparations with various purposes are currently used in agriculture. There are among them growth-stimulating preparations as well as preparations suppressing the development of phytopathogenic bacteria and fungi. Microorganisms can stimulate plant growth (Azospirillum), bind nitrogen (Rhizobium), prevent plant diseases (Pseudomonas, Bacillus) or destroy insect pest (Streptomyces).

“Seed treatment method” described in application no. 2170987 of Great Britain, priority date 14.02.85, IPC 4A01C1/06, published in Izobreteniya stran mira (Inventions of countries across the world) no. 10 in 1987 is known. According to it, seeds are treated with a mixture of microorganisms, a carrying agent such as bran and glue such as gum ghatti. Treatment of wheat seeds yields the best results.

Russian Federation patent no. 2140138 for a group of inventions “Method of pre-sowing treatment of seeds of vegetable crops and method of obtaining preparation for pre-sowing treatment of seeds of vegetable crops” is known, application no. 98120341, priority date 13 Nov. 1998, registered in the name of ZAO SSPR SORTSEMOVOSHCH, IPC A01C1/06, publication date 27 Oct. 1999.

The above methods are based on a biofungicidal preparation containing the bacterial strain Bacillus subtilis CH-13 (deposited under the registration number of ARRIAM D-606 in the group of epiphytic microorganisms). The biofungicidal preparation is produced by mixing cultural liquid containing strain Bacillus subtilis CH-13 previously cultivated in liquid sterile nutrient medium, with PVA emulsion, water dissolvent and sterile chalk or dolomite in certain proportions. The invention allows one to increase the effectiveness of protection of vegetable crops from phytopathogenic fungi by pre-sowing treatment of seeds.

Russian Federation patent no. 2099947 for the invention “Phytosporin biopreparation for the protection of plants from diseases”, application 96121980, is known; priority date 15 Nov. 1996, registered in the name of Institute for Microbiology and Virusology of the National Academy of Sciences of the Ukraine (UA) and NPO Bashkirskoye (RU), IPC A01N63/00; C12N1/20; C12R1:125, publication date 27 Dec. 1997.

Phytosporin biopreparation is based on the bacterial strain Bacillus subtilis ARRIAM 128 with the cell concentration of 10⁹-10¹⁰ per 1 ml of physiological solution in the amount of 92-98 vol. % and filler in the amount of 2-8 vol. %. The strain Bacillus subtilis ARRIAM 128 is characterised by a high antagonistic activity against phytopathogenic bacteria and fungi and therefore can be used for protection of various agricultural plants (grain and legume crops) and ornamental trees by pre-sowing seed treatment.

Russian Federation patent no. 2478290 for the invention “Biopreparation for stimulating plant growth, protecting plants from diseases, increasing yield and soil fertility” is known, RF patent no. 2478290 (application no. 2011145665), priority date 11 Nov. 2011, registered in the name of OOO Batsiz, IPC A01N63/02, A01C1/06, C12N1/20, C12R1/07, application publication date 20 May 2012, patent publication date 10 Apr. 2013.

The biopreparation contains the biomass of Bacillus amyloliquefaciens RNCIM B-11008 and humates in the following proportions, in vol. %: biomass of vegetating cells and spores of Bacillus amyloliquefaciens RNCIM B-11008 1.24÷1.30×10¹⁰ CFU/ml of the cultural fluid and spore content 94% out of the total CFU 99.0, humates 1.0. The biopreparation can be used to protect plants from fungal and bacterial diseases, improve phytosanitary conditions in the soil, increase soil fertility and yield of crops.

The disadvantages of the preparation are a limited application area (only wheat and barley) and the need to ensure a high titre of cells and spores of the producing strain for the preparation to be effective.

Russian Federation patent no. 2528058 for the invention “Bacterial strain Bacillus amyloliquefaciens with a fungicidal and bactericidal effect and biological preparation on its basis for the protection of grain plants from diseases caused by phytopathogenic fungi” is known; application 2013125726, priority date 4 Jun. 2013, registered in the name of FGUP GosNIIgenetika, IPC A01N63/02, A01C1/06, C12N1/20, C12R1/07, publication date 10 Sep. 2014.

A bacterial strain Bacillus amyloliquefaciens RNCIM B-11475 with a fungicidal and bactericidal effect was described and a biological preparation for the protection of grain plants from diseases caused by phytopathogenic fungi was proposed. The biological preparation is obtained by mixing the active agent (cultural liquid of the strain in question with a titre of 2-3×10⁹ CFU/ml and a carrying agent in the form of fine diatomite pellets) in the volume proportion of 1:3 with subsequent drying.

The inventions can be used to increase the yield of grain crops and decrease the prevalence of phytopathogenic fungi.

Its disadvantages are a limited application area (only grain crops) and the lack of bactericidal activity.

Patent application of China CN102703354 (A) of 3 Oct. 2012 for bacterial strain Bacillus amyloliquefaciens subsp. plantarum B203 with a fungicidal effect against anthracnose of strawberry and some other fungal diseases is known; IPC A01N63/00, -/02; A01P3/00; C12N1/20; C12R1/07, and patent application of China CN104195072 (A) of 10 Dec. 2014 based on the national application CN20141385641, priority date 6 Aug. 2014, for bacterial strain Bacillus amyloliquefaciens subsp. plantarum B232 with a fungicidal effect against bark canker of poplar and some other fungal diseases; IPC A01N63/00; A01P3/00; C12N1/20; C12R1/07.

The disadvantages of these strains are a limited application area (strawberry, poplar) and the lack of bactericidal activity. Besides, the patents do not mentioned the titres necessary for the preparation to be effective for the control of fungal diseases of strawberry and poplar.

Patent application of Romania R0127468 (A2) of 29 Jun. 2012 based on the national application R020100001379 is known, priority date 21 Dec. 2010 for the invention “Strain Bacillus amyloliquefaciens subsp. plantarum B100 for growth of agricultural crops”, IPC A01N63/00; C12N1/20.

The strain has a fungicidal (production of lipopeptide and polyketide antibiotics) and bactericidal effect against a wide range of phytopathogenic fungi in the soil and bacterial diseases of fruit trees as well as a stimulating effect based on endophytic production of growth factors. It can produce a number of enzymes such as proteases, lactonases, amylases, phytases and cellulases and dissolve inorganic compounds of phosphorus and selenium. The strain can be used for strengthening the grain of grain crops (wheat and maize) in regions with selenium deficiency.

The publication does not mention the titre of Bacillus amyloliquefaciens subsp. plantarum B100 necessary in the preparation when the strain is used for control of plant diseases. Another disadvantage is its limited application area (grain crops and fruit trees).

An international application WO2012130221 (A2) of 24 Mar. 2012 based on the national application of Germany DE20111015803 is also known, priority date 1 Apr. 2011 for the invention “Product against phytopathogenic microorganisms”, IPC A01N63/00; C07K14/32; C12N1/20; C12R1/07. A new strain Bacillus amyloliquefaciens subsp. plantarum AB101 was described, which is highly effectiveness against root rot of potato (black scab) caused by Rhizoctonia solani.

An antifungal preparation Bacteriocin was developed for the treatment of fungal, other microbial and viral infections. It is based on spores of bacteria Bacillus amyloliquefaciens subsp. plantarum AB101 with a strong effect against Gram-positive bacteria.

The strain Bacillus amyloliquefaciens subsp. plantarum produces ten various substances: fungicidal (various groups of antibiotic substances such as dipeptides, lipopeptides and siderophores), bactericidal (polyketides and bacteriocins) and antiviral substances against a broad range of plant diseases, especially root rot of potato (black scab) caused by the phytopathogenic fungus Rhizoctonia solani. It can be used in agriculture, plant protection and biotechnology.

The publication does not mention the titre of Bacillus amyloliquefaciens subsp. plantarum AB101 necessary in the preparation when the strain is used for control of plant diseases.

Another disadvantage is a limited application area: the effect against potato root rot caused by the phytopathogenic fungus Rhizoctonia solani was mostly described.

Patent no. 2495119 for the invention “Bacterial strain Bacillus subtilis 8A as a means of increasing plant productivity and plant protection from phytopathogenic microorganisms”, application no. 2012151104, is known, priority date 29 Nov. 2012, registered in the name of GNU ARRIAM of Rossel'khozakademiya. This strain was chosen as a prototype.

### SUMMARY OF THE INVENTION

The strain is deposited in the collection of the All-Russia Research Institute of Agricultural Microbiology (ARRIAM) of Rossel'khozakademiya on 14 Nov. 2011 under the number RCAM 00876 as a means of increasing plant productivity and plant protection from phytopathogenic microorganisms. This strain has a high fungicidal and bactericidal activity. This strain and the biopreparation on its basis were also shown to have a high growth-stimulating activity, which results in an increased yield. Experiments confirmed that the effectiveness of the strain is due to the ability of its bacteria to form a microbial-plant system by colonising the rhizosphere and the roots of plants.

Bacterial strains from the taxonomic group Bacillus amyloliquefaciens subsp. plantarum are deposited in collections situated far from the applicant (some of them in other countries). Moreover, some publications do not mention the titre of the bacterial strain necessary for its use in the biopreparation. For these reasons, the bacterial strain Bacillus subtilis 8A, which is deposited in the collection of the All-Russia Research Institute of Agricultural Microbiology (ARRIAM) and is available to the applicant, was chosen as a prototype. The object-matter of the invention is isolation of a strain of rhizosphere bacteria suitable for use in agriculture as a means of plant protection against phytopathogenic microorganisms, improvement of nutrition of agricultural crops and increasing plant productivity, in this way broadening the toolkit of such products.

The claimed solution is the use of the strain of rhizosphere bacteria Bacillus amyloliquefaciens subsp. plantarum BS89 as a means of increasing plant productivity and protecting plants from phytopathogenic microorganisms.

The strain of rhizosphere bacteria Bacillus amyloliquefaciens subsp. plantarum BS89 was isolated from the roots of winter wheat, cv. Lira, growing on chernozem soils of Krasnodar Territory (Russian Federation).

The strain is deposited in the Russian Collection of Agricultural Microorganisms (RCAM) of ARRIAM, deposition date 9 Jul. 2015, under the number RCAM 03458 as a means of increasing plant productivity and their protection from phytopathogenic microorganisms (a copy of the deposition certificate is attached).

### DESCRIPTION OF PREFERRED EMBODIMENTS OF THE INVENTION

The strain has the following morphological-cultural and physiological-biochemical characteristics.

Bacterial cells have the shape as regular rods with rounded ends. The arrangement of flagella is monopolar, peritrichal. Cell size (0.9-1.8) am. The bacteria form spores located in the centre of the cell and stain positively after Gram. After 24 hours of growth, accumulation of poly-β-hydroxybutyrate was observed in liquid nutrient medium.

Growth in liquid and semiliquid nutrient medium is microaerophilic, metabolism is respiratory and fermentative. On beef extract agar, the strain forms dry cream-coloured colonies with a paste-like texture and uneven rugged edges. The diameter of the colonies is (5-12) mm. Optimal growth temperature is 33° C. Growth slows down at a temperature more than 45° C. or less than 15° C. Optimal pH value 6.8, pH from 4.5 to 9.0 is also suitable for growth. The strain hydrolyses casein, gelatine, starch and litmus milk, with litmus loosing colour in the process. It has a strong catalase, amylase, protease, lipase and phospholipase activity. It can grow at 50° C., 10% NaCl and 0.001% lysozyme.

As the only source of carbon, the strain uses (with formation of acid) arabinose, xylose, mannitol, glucose, galactose, fructose, maltose, sorbitol, glycerine, dextrin, starch and (with formation of alkali) rhamnose and dulcite. It mostly utilises mineral forms of nitrogen (ammonium salts and nitrates) as well as amino acids and proteins. Experiments showed that bacterial strain Bacillus amyloliquefaciens subsp. plantarum BS89 increases the yield of plants and has antagonistic properties against several pathogens of agricultural crops such as:
in case of winter and spring wheat, against mildew (Erysiphe graminis), brown rust (Puccinia recondita), fusariose root rot (Fusarium culmorum) and basal glume rot (Pseudomonas syringae);
in case of spring barley, against seed mould (Penicillium, Alternaria), root rot (Bipolaris sorokiniana) and brown spot (Drechslera sorokiana);
in case of white cabbage and cauliflower, against black rot (Xanthomonas campestris), black stem (Rhizoctonia solani), Pythium root rot (Pythium irregulare);
in case of potato, against late blight (Phytophthora infestans), Rhizoctonia blight (Rhizoctonia solani) and Fusarium wilt (Fusarium oxysporum);
in case of sugar beet, against diseases caused by fungi Pythium debaryanum and Phoma betae and against Cercospora blight (Cercospora beticola);
in case of sunflower, against white rot (Sclerotinia sclerotiorum) and stem blight (Phomopsis helianthi);
in case of flax, against Fusarium wilt (Fusarium avenaceum Sacc, Fusarium oxysporum v. orthoceros f. lini (Boll) Bilai) and bacterial blight (Clostridium macerans Schard).

Experiments showed that bacterial strain Bacillus amyloliquefaciens subsp. plantarum BS89 has a fungicidal activity against phytopathogenic fungi Fusarium culmorum, Fusarium graminearum, Fusarium sporotrichioides, Erysiphe graminis, Phytophtora infestans, Rhizoctonia solani, Pythium irregular, Plasmopara viticola and Uncinula necator, Botrytis cinerea and bactericidal activity against phytopathogenic bacteria Xanthomonas campestris, Pseudomonas syringa and Clavibacter michiganense.

Four strains of rhizosphere bacteria were tested in respect of their antagonistic activity: Pseudomonas fluorescens AP-33 (microbial preparation Planrhiz), Bacillus subtilis ARRIAM 128 (microbial preparation Phytosporin), Bacillus subtilis 8A (prototype) and Bacillus amyloliquefaciens subsp. plantarum BS89 (claimed strain) The results are presented in Tables 1 and 2.

Bactericidal activity was tested on five strains of phytopathogenic bacteria: Pseudomonas syringae 8300, Pseudomonas syringae 2314, Erwinia carotovora A-1, Erwinia carotovora 3391 and Clavibacter michiganense 17-1.

At first, bacteria were inoculated with the help of bacteriological loop as a lawn on the surface of nutrient agar and cultured for 48 hours at 28° C. On the day of the test for bactericidal activity, strains of phytopathogenic bacteria used in the study were inoculated as a lawn onto starvation potato agar. Sterile agar blocks with a mature culture of the tested bacteria were cut out with the help of a drill bit and were transferred to the surface of freshly inoculated medium with the help of a sterile scalpel and flame-sterilized pincers. Petri dishes with blocks were inoculated for 24 hours at 28° C., after which the diameter of the inhibition zone around the blocks was measured. The results of measurements are presented in Table 1.

TABLE 1
Antagonistic. Growth inhibition zone, mm.

| activity of the strains | | | | | |
|---|---|---|---|---|---|
| of rhizosphere | | | | | |
| bacteria against | | | | | |
| phytopathogenic | Erwinia | Pseudomonas | Pseudomonas | Erwinia | Clavibacter |
| bacteria | carotovora | syringae | syringae | carotovora | michiganense |
| Bacterial strain | A-1 | 8300 | 2314 | 3391 | 17-1 |
| Pseudomonas | 11.0 + 0.8 | 7.5 + 0.3 | 11.3 + 1.0 | 12.5 + 1.1 | 15.6 + 1.1 |
| fluorescens AP-33 | | | | | |
| (Planrhiz) | | | | | |
| Bacillus subtilis | 9.8 + 0.7 | 11.0 + 0.8 | 12.5 + 1.0 | 17.5 + 1.2 | 17.7 + 1.3 |
| ARRIAM 128 | | | | | |
| (Phytosporin) | | | | | |
| Bacillus subtilis 8A | 17.5 + 1.3 | 25.5 + 1.9 | 27.7 + 2.0 | 24.1 + 1.7 | 22.1 + 1.7 |
| (prototype) | | | | | |
| Bacillus | 20.5 + 1.7 | 35.1 + 2.5 | 49.7 + 3.7 | 55.3 + 3.9 | 42.3 + 2.9 |
| amyloliquefaciens | | | | | |
| subsp. plantarum | | | | | |
| BS89 (claimed) | | | | | |

Data in Table 1 indicate that the claimed bacterial strain Bacillus amyloliquefaciens subsp. plantarum BS89 has a greater antagonistic activity against phytopathogenic bacteria than the prototype strain Bacillus subtilis 8A.

Fungicidal activity was tested against five phytopathogenic fungi: Phytophtora infestans, Rhizoctonia solani, Fusarium culmorum, Fusarium solani, Pythium ultimum using the method of wells.

A suspension of fungal spores (10⁵ CFU/ml) was added to potato-dextrose agar, heated and cooled down to 37° C., at a rate of 1 mcl of suspension per 1 ml of the medium. The mixture was poured into Petri dishes. After congelation, four smooth through holes were made in the mixture with the help of a flame-sterilized drill bit. The holes were arranged at the vertices of a square. One hundred mcl of the bacterial suspension with a cell titre of 10⁸ CFU/ml was poured Into each hole. One Petri dish with empty wells served as a control. All dishes with wells were cultured for 72 hours at 28° C. Fungicidal activity was determined as the size of zones around the wells where the growth of the phytopathogenic fungus was inhibited. The results of measurements are presented in Table 2.

TABLE 2
Antagonistic activity of the strains of rhizosphere
bacteria against phytopathogenic fungi.

| | Growth inhibition zone, mm | | | | |
|---|---|---|---|---|---|
| | Phytophtora | Rhizoctonia | Fusarium | Fusarium | Pythium |
| Bacterial strain | infestans | solani | culmorum | solani | ultimum |
| Pseudomonas | 3.5 + 0.2 | 11.1 + 0.7 | 7.5 + 0.5 | 12.8 + 0.9 | 12.5 + 1.1 |
| fluorescens AP-33 | | | | | |
| (Planrhiz) | | | | | |
| Bacillus subtilis | 11.1 + 0.8 | 13.5 + 1.0 | 19.5 + 1.6 | 21.4 + 1.5 | 25.3 + 2.0 |
| ARRIAM 128 | | | | | |
| (Phytosporin) | | | | | |
| Bacillus subtilis | 21.3 + 1.9 | 19.5 + 1.1 | 23.5 + 1.5 | 25.3 + 2.0 | 27.7 + 2.1 |
| 8A (prototype) | | | | | |
| Bacillus | 28.7 + 1.9 | 35.8 + 2.9 | 37.3 + 2.9 | 30.7 + 1.8 | 45.3 + 3.1 |
| amyloliquefaciens | | | | | |
| subsp. plantarum | | | | | |
| BS89 (claimed) | | | | | |

Data in Table 2 indicate that the claimed bacterial strain Bacillus amyloliquefaciens subsp. plantarum BS89 has a higher antagonistic activity against phytopathogenic fungi than the prototype strain Bacillus subtilis 8A.

An analysis of whole-genome sequencing showed that this activity of the strain Bacillus amyloliquefaciens subsp. plantarum BS89 was due to the presence of genes encoding the production of fungicidal and bactericidal substances such as surfactins, fengycins, bacillomycin D, cyclic lipopeptides, bacillibactin, bacillolysin, bacillaene, macrolactin, plantazolicin and amylocyclizin (Table 3).

Non-ribosomal peptides (NRPs) are a broad range of structurally different antibiotic substances. The best known and best studied NRPs are surfactins, fengycins and iturins.

Iturins and fengycins are the major factors underlying antifungal activity of various bacilli [1].

Fengycins are especially active against mycelial fungi [2].

Surfactins are necessary for the formation of biofilms and the dispersal of bacteria in the environment, in this way promoting the colonization of plant roots and tissues and the manifestation of biocontrol activity [3]. Besides, surfactins and, to a lesser extent, fengycins can trigger protective mechanisms in plants [1].

TABLE 3
Gene clusters involved in the synthesis of antibiotic metabolites of
the strain Bacillus amyloliquefaciens subsp. plantarum BS89.

| Class of | | | |
|---|---|---|---|
| substances | Metabolite | Gene cluster | Biological function |
| Non-ribosomal | Surfactins | srfABCD | Formation of biofilms, |
| peptides | | | antifungal activity |
| | Fengycins | fenABCDE | Antifungal activity |
| | Bacillomycin | bmyCBAD | Antifungal activity |
| | Cyclic | nrsABCDEF | Unknown |
| | lipopeptides | | |
| | Bacillibactin | dhbABCDEF | Synthesis of |
| | | | siderophores |
| | Bacilysin | bacABCDE | Antibacterial activity |
| Polyketides | Dificidin | dfnAYXBCDEFG | Antibacterial activity |
| | Bacillaene | baeBCDEGHIJL | Antibacterial activity |
| | Macrolactin | mlnABCDEFGHI | Antibacterial activity |
| Small ribosomal | Plantazolicin | pznFKGHIAJCD | Nematicidal and |
| peptides | | | antibacterial activity |
| | Amylocyclizin | acnBACDEF | Antibacterial activity |
| | | | (against Gram-positive |
| | | | bacteria) |

Besides lipopeptides, the gene cluster dhb is involved in the synthesis of a siderophore bacillibactin. Bacterial siderophores have a high affinity to ferric iron and can effectively bind it in iron-deficient environments such as soils. As a result, iron ions become less available to phytopathogens, and the biocontrol activity of siderophore-producing bacteria is promoted in this way (Kloepper et al., 1980). Operon dhb responsible for the synthesis of bacillibactin is similar to the corresponding operon in Gram-negative bacteria producing enterobactin.

Besides lipopeptides, the gene cluster dhb is involved in the synthesis of a siderophore bacillibactin. Bacterial siderophores have a high affinity to ferric iron and can effectively bind it in iron-deficient environments such as soils. As a result, iron ions become less available to phytopathogens, and the biocontrol activity of siderophore-producing bacteria is promoted in this way [4]. Operon dhb responsible for the synthesis of bacillibactin is similar to the corresponding operon in Gram-negative bacteria producing enterobactin.

Bacillus amyloliquefaciens subsp. plantarum BS89 has three large clusters of genes encoding polyketides dificidin, bacillaene and macrolactin (Table 3). All these three metabolites have a broad antibacterial activity against plant and human pathogens and potentially may be used in medicine [5, 6].

The genome of the strain Bacillus amyloliquefaciens subsp. plantarum BS89 contains a gene cluster for the production of two small ribosomal peptides, plantazolicin and amylocyclizin (Table 3). Plantazolicin belongs to a new type of small ribosomal peptides with a narrow antimicrobial activity against other bacilli, especially B. anthracis (anthrax pathogen) [7]. Besides, this metabolite can suppress nematodes, which form galls on plant roots. Several other Gram-positive bacteria, including Clavibacter michiganensis subsp. sepedonicus, Corynebacterium urealyticum DSM7109 and Brevibacterium linens BL2, have a similar biosynthetic cluster in their genomes [8].

Amylocyclizin is a cyclic peptide from the group of bacteriocins. It has a high antibacterial activity against closely associated Gram-positive bacteria. This advantage can be used for the suppression of bacterial competitors in the rhizosphere [7]. Gene cluster acn responsible for the synthesis of this bacteriocin is common among species from Bacillus/Paenibacillus taxonomic groups. Another small peptide, mersacidin, was also found in several strains of Bacillus amyloliquefaciens subsp. plantarum [9] but the strain BS89 contains only fragments of the entire gene cluster and may not be able to synthesize mersacidin.

Experiments showed that the bacterial strain Bacillus amyloliquefaciens subsp. plantarum BS89 does not only have a fungicidal activity against phytopathogenic bacteria and phytopathogenic fungi but also exercises a phytostimulating effect on various agricultural crops such as radish and wheat.

Growth-stimulating activity of the bacterial strain was analysed with the help of an original technique and the use of radish plants, cv. Duro, and wheat, cv. Veda. Seeds were sterilized for 2 minutes in 70% ethanol and rinsed in sterile tap water. Then they were soaked for 30 min in a bacterial suspension with a titre of 10⁷ CFU/ml and put into sterile moist chambers, 20 seeds into each, in 3 replications. Control seeds were soaked in sterile tap water. Then the plants were incubated in the phytotron for 72 hours at 28° C. After incubation, the length of rootlets and shoots was measured. Growth-stimulating activity of the tested bacterial strains was calculated as compared to control seedlings. The results are presented in Tables 4 and 5.

TABLE 4
Growth-stimulating activity of the strain of
rhizosphere bacteria on radish seedlings.

| | | Average length of |
|---|---|---|
| | Average root | the green part of |
| Experimental variant | length, mm | the seedling, mm |
| Control (without treatment) | 12.1 ± 0.7 | 19.5 ± 1.3 |
| Treatment with Bacillus | 15.3 ± 1.0 | 25.0 ± 2.1 |
| subtilis 8A (prototype) | | |
| Treatment with Bacillus | 23.5 ± 1.5 | 29.7 ± 2.3 |
| amyloliquefaciens subsp. | | |
| plantarum BS89 (claimed) | | |

Data in Table 4 indicate that the claimed bacterial strain Bacillus amyloliquefaciens subsp. plantarum BS89 has a stronger growth-stimulating activity on radish seedlings than the prototype strain Bacillus subtilis 8A.

TABLE 5
Growth-stimulating activity of the strain
of rhizosphere bacteria on wheat seedlings.

| | | Average length of |
|---|---|---|
| | Average root | the green part of |
| Experimental variant | length, mm | the seedling, mm |
| Control (without treatment) | 9.3 ± 0.5 | 13.7 ± 1.0 |
| Treatment with Bacillus | 13/5 ± 1.1 | 16.9 ± 1.3 |
| subtilis 8A (prototype) | | |
| Treatment with Bacillus | 19.9 ± 1.2 | 22.1 ± 1.7 |
| amyloliquefaciens subsp. | | |
| plantarum BS89 (claimed) | | |

Data in Table 5 indicate that the claimed bacterial strain Bacillus amyloliquefaciens subsp. plantarum BS89 has a stronger growth-stimulating activity on wheat seedlings than the prototype strain Bacillus subtilis 8A.

A high growth-stimulating activity of the bacterial strain Bacillus amyloliquefaciens subsp. plantarum BS89 is presumably due to its ability to produce a number of vitamins such as thiamine (B1), riboflavin (B2), nicotinate (B3), pantothenate (B5), biotin (B7), folate (B9), cobalamin (B12) and menaquinone (K2) (Table 6). Vitamins are indispensable elements of nutrition produced with the help of various plants and bacteria [10]. Their major physiological role is serving as cofactors in numerous metabolic processes and as antioxidants.

Out of eight known B vitamins, six were found in the genome of Bacillus amyloliquefaciens subsp. plantarum BS89 annotated with KEGG ontology (using KAAS server) and genome data mining (Table 6). They are thiamine (B1), riboflavin (B2), pantothenate (B5), vitamin B6 (pyridoxin), biotin (B7) and folate (B9).

Besides, we found a protein involved in the synthesis of cobalamin (vitamin B12) (Prokka_00310) and two enzymes involved in the metabolism of nicotinate (vitamin B3): nicotinate-phosphoribosyl transferase (Prokka_02867) and nicotinate nucleotide pyrophosphorylase (Prokka_02473). Dimethyl metaquinon methyltransferase (Prokka_02063) was also found in the genome of Bacillus amyloliquefaciens subsp. plantarum BS89. This enzyme catalyses the last step in the biosynthesis of K2 vitamin. These results indicate that Bacillus amyloliquefaciens subsp. plantarum BS89 may be capable of producing vitamins B3, B12 and K2.

TABLE 6
Vitamins in the genome of Bacillus amyloliquefaciens
subsp. plantarum BS89 and their biological role.

| | | Possible role in plant-microbial |
|---|---|---|
| Vitamin type | Functions in the bacterium* | interactions** |
| Thiamine | Decarboxylation of keto acids | Cofactor of indole-3-pyruvate |
| (B1) | and transaminase reactions | decarboxylase (biosynthesis of indole |
| | | acetic acid), induction of systemic |
| | | stability of plants, stimulating action on |
| | | nodule bacteria |
| Riboflavin | Oxidation-reduction reactions | Induction of systemic stability in plants, |
| (B2) | | stimulation of plant growth, activation |
| | | of quorum-sensing genes in bacteria |
| Nicotinate | Nicotine amide adenine | Reduction of salinity stress, protective |
| (B3) | dinucleotide (NAD) | mechanisms |
| Pantothenate | Oxidation of keto acids and | Unknown |
| (B5) | carriers of acyl groups | |
| Pyridoxin | Transamination, deamination, | Protection against osmotic and |
| (B6) | decarboxylation and | oxidative stress |
| | racemation of amino acids | |
| Biotin (B7) | Biosynthetic reactions | Stimulation of bacterial growth and |
| | requiring CO2 fixation | colonization |
| Folate (B9) | Transfer of one-carbon units | Unknown |
| | necessary for the synthesis of | |
| | thymine, purine bases, serine, | |
| | methionine and pantothenate | |
| Cobalamin | Transfer of methyl groups | Formation of legume-rhizobial |
| (B12) | | symbiosis |
| Menaquinone | Transport of electrons | Unknown |
| (κ2) | | |

*Source: an online bacteriology manual http://textbookofbacteriology.net/nutgro_2.html
**Source: a review by Palacios et al. (2014)

An example of production of a microbial preparation based on the claimed strain of rhizosphere bacteria Bacillus amyloliquefaciens subsp. plantarum BS89 in liquid form is given below.

Stock Culture

To obtain stock culture of the bacterial strain Bacillus amyloliquefaciens subsp. plantarum BS89, liquid nutrient medium PSB (potato-saccharose broth) is used. Potato broth is first made by cutting 200 g of peeled potato into slices and boiling it in 800 ml of distilled water for 20 minutes. The broth is then filtered through a cotton-gauze filter, 10 g of saccharose is added and the pH of the mixture is made to achieve 7.0.

The obtained liquid nutrient medium is poured into 750 ml shake flasks, 100 ml into each flask, and sterilised for 30 minutes at 1 atm. The nutrient medium is then inoculated in the flasks at a rate of: 1 tube with slanted nutrient agar containing a pure culture of Bacillus amyloliquefaciens subsp. plantarum BS89 per one flask. After that, the flasks are placed in a shaker (180 rpm) and cultured for 48 hours at 28° C. In this way, stock culture Bacillus amyloliquefaciens subsp. plantarum BS89 is obtained in flasks with a titre of bacteria ca. 4×10⁹ CFU/ml. It is stored in the fridge up to one month at 4-6° C. for subsequent inoculation of fermenters.

Reference Culture

A reference culture of the bacterial strain Bacillus amyloliquefaciens subsp. plantarum BS89 for industrial culturing is obtained in fermenters on the medium with molasses and maize extract. Inoculation rate of the stock culture is 1-3%, the time of cultivation is 72 hours at 33° C. During cultivation of reference cultures, the temperature rise up to 37° C. is acceptable. In this way, a concentrated bacterial suspension based on strain Bacillus amyloliquefaciens subsp. plantarum BS89 with a titre of bacteria not less than 1×10⁹ CFU/ml is obtained. Liquid form of the microbial preparation based on strain Bacillus amyloliquefaciens subsp. plantarum BS89.

A concentrated bacterial suspension based on the strain Bacillus amyloliquefaciens subsp. plantarum BS89 is diluted with sterile water in the proportion 1:10 or 1:20 depending on the titre of the concentrated bacterial suspension. The obtained liquid form is kept for 3-5 days at 20-25° C. until the bacterial titre of at least 1×10⁸ CFU/ml is obtained. After that, the microbial preparation is ready for use in agriculture. Liquid preparation is poured under sterile conditions into polythene bottle or canisters rinsed with alcohol.

Shelf life of the preparation is at least 24 months. Experiments showed that for the strain Bacillus amyloliquefaciens subsp. plantarum BS89 to be effective in various microbial preparations, the concentration (the amount of viable cells and spores) of its bacteria should be 10⁴-10⁹ cells in 1 ml of the culture liquid. The use of the strain in a concentration of less than 10⁴ cells/ml decreases the antagonistic and growth-stimulating effect, while an increase of the concentration above 10⁹ cells/ml does not enhance it.

The effectiveness of the microbial preparation based on the claimed strain of rhizosphere bacteria Bacillus amyloliquefaciens subsp. plantarum BS89 obtained as described above was tested in greenhouse trials on winter wheat, cv. Veda, and radish, cv. Duro Krasnodarskoye.

For these trials, pots with a volume of 3 l (for wheat and for radish) were stuffed with soil up to the weight of 3.4 kg. Before sowing, the soil was wetted until full field moisture retaining capacity with tap water with a volume of 200 ml. Radish and wheat seeds were sorted by size, sterilised and germinated in sterile Petri dishes with sterile filter paper.

Similarly sized seedlings were divided into three parts. One part of the seedlings was inoculated for 30 min in a liquid preparation based on the prototype strain Bacillus subtilis 8A with a titre of 10⁷ CFU/ml. The second part was inoculated in the preparation based on the claimed strain Bacillus amyloliquefaciens subsp. plantarum BS89 with a titre of 10⁷ CFU/ml and obtained as described above. The third part of the seedlings was treated with sterile water (control). The biomass of radish plants was measured after 25 days, and that of wheat plants, after 30 days.

The results of trials with are presented in Tables 7 and 8.

TABLE 7
Effectiveness of the strain of rhizosphere bacteria
in greenhouse trials on wheat, cv. Veda

| Experimental variant: treatment with | | Added biomass as compared | |
|---|---|---|---|
| biopreparation based on the bacterial | | to control | |
| strain | Biomass, g | g | % |
| Control (without treatment) | 85 | — | — |
| Bacillus subtilis 8A (prototype) | 101 | 16 | 18.8 |
| Bacillus amyloliquefaciens subsp. | 122 | 37 | 43.5 |
| plantarum BS89 (claimed) | | | |

TABLE 8
Effectiveness of the strain of rhizosphere bacteria in
greenhouse trials on radish, cv. Duro Krasnodarskoye.

| Experimental variant: treatment with | Biomass | | Weight of radish | |
|---|---|---|---|---|
| biopreparation based on the bacterial | | % added to | | % added to |
| strain | g | the control | g | the control |
| Control (without treatment) | 42.5 | — | 18.0 | — |
| Bacillus subtilis 8A (prototype) | 50.2 | 18.1 | 29.1 | 61.7 |
| Bacillus amyloliquefaciens subsp. | 65.3 | 53.6 | 38.5 | 113.9 |
| plantarum BS89 (claimed) | | | | |

The comparison of the data obtained in greenhouse trials on wheat, cv. Veda, and radish, cv. Duro Krasnodarskoye (Table 7 and 8) indicate that the microbial preparation based on the claimed bacterial strain Bacillus amyloliquefaciens subsp. plantarum BS89 has a greater effect on wheat and radish than the microbial preparation based on the prototype strain Bacillus subtilis 8A.

Experiments showed that the use of 10% solution of the microbial preparation based on the claimed strain of rhizosphere bacteria Bacillus amyloliquefaciens subsp. plantarum BS89 is optimal for the treatment of seeds of agricultural plants, while 0.1-3% solution is optimal for vegetating plants.

The effectiveness of the strain Bacillus amyloliquefaciens subsp. plantarum BS89 for increasing productivity and quality was tested in field trials on grain, vegetable and industrial crops (spring and winter wheat, barley, potato, flax, sunflower and sugar beet) with the application of three microbial preparations: Phytosporin based on bacterial strain Bacillus subtilis ARRIAM 128, microbial preparation based on the prototype strain Bacillus subtilis 8A and microbial preparation based on the claimed bacterial strain Bacillus amyloliquefaciens subsp. plantarum BS89 obtained as described above.

A field trial with spring wheat, recognized cv. Lada, was made in Krasnodar Territory on powerful, medium loamy, medium humic chernozem soil, humus after Tyurin 3.24%, nitrate nitrogen 8.3 mg/kg, phosphorus 64 mg/kg, potassium 150 mg/kg, aqueous pH 6.46.

The field trial was conducted following the recommendations of the Geographic Trial Network of the Pryanishnikov All-Russia Research Institute for Agrochemistry (Assessment of effectiveness of microbial preparations in agriculture/Ed. A. A. Zavalin, Moscow: Rossel'khozakademiya, 2000, 82 p.). Sowing plot area 40 m², record plot area 30 m². The trial was conducted in four replications. The land was previously left to lie fallow. The background of the fertilizer was N₆P₅₂ (ammophos). Pre-sowing treatment: cultivation at a depth of 5-7 cm. Seeding rate of spring wheat: 5.0 million fertile seeds per hectare.

On the day of sowing, a part of wheat seeds was left untreated (control), the second part was treated with a liquid biopreparation Phytosporin based on the strain Bacillus subtilis 128 ARRIAM (standard), the third part of seeds was treated with liquid biopreparation based on the strain Bacillus subtilis 8A (prototype), and the last part of seeds was treated with the preparation based on the bacterial strain Bacillus amyloliquefaciens subsp. plantarum BS89 obtained as described above. Wheat seeds were treated at a rate of 1 l of the preparation per 9 l of water with the addition of 20-30 g of sodium carboxymethyl cellulose.

The soil was cultivated before sowing. For this, mineral fertilizers were manually applied to it in the form of ammophos (N₆P₅₂) according to the experimental scheme. After that, wheat seeds were sown into the sowing zones delimited according to the biopreparation used for treatment as well as the control zone.

Foliage spraying of plants was conducted during the phase of 3-5 leaves and in the phase of the end of tillering/beginning of booting. Plants were sprayed with the help of a shoulder sprayer at a rate of 1 l of the preparation per 300 l of water per hectare.

The farming culture of wheat corresponded to the zonal technology. The yield was harvested with the help of SAMPO-130 harvester and recorded totally, plot-by-plot. It was recalculated to 100% purity and 14% humidity. Gluten content was determined based on GOST 13586.1/68, and protein content, based on GOST 10846/91. Statistical assessment of the significance of the obtained results was based on dispersion analysis with a 95% significance level. The results of trials with spring wheat are presented in Table 9.

TABLE 9
Effect of the strain of rhizosphere bacteria on the yield and
grain quality of spring wheat, cv. Lada (Krasnodar Territory).

| | Grain | | | | | |
|---|---|---|---|---|---|---|
| | yield, | Added yield | | Grain quality | | |
| | 100 | 100 | | Gluten | FDM, | Grain |
| Variant | kg/ha | kg/ha | % | % | units | class |
| Control | 39.0 | — | — | 22.5 | 85 | 3 |
| Bacillus subtilis | 42.5 | 4.3 | 11.0 | 23.5 | 80 | 3 |
| ARRIAM 128 | | | | | | |
| (Phytosporin, standard) | | | | | | |
| Bacillus subtilis 8A | 45.1 | 6.1 | 15.6 | 24.0 | 80 | 3 |
| (prototype) | | | | | | |
| Bacillus | 49.7 | 10.7 | 27.4 | 26.0 | 75 | 2 |
| amyloliquefaciens | | | | | | |
| subsp. plantarum BS89 | | | | | | |
| (claimed) | | | | | | |

A field trial with winter wheat, cv. Mironovskaya 808, was made in the Ulyanovsk Region on bleached, medium powerful, loamy, medium humic chernozem soil, humus after Tyurin 2.89%, nitrate nitrogen 9.5 mg/kg, phosphorus 79 mg/kg, potassium 140 mg/kg, aqueous pH 6.62.

Sowing plot area was 30 m², record plot area was 25 m². The trial was conducted in four replications. The land was previously left to lie fallow. The background of the fertilizer was N₆P₅₂ (ammophos). Pre-sowing treatment: cultivation at a depth of 5-7 cm. Application rate of winter wheat: 4.8 million fertile seeds per hectare.

On the day of sowing, a part of seeds wheat was left untreated (control), the second part was treated with a liquid biopreparation Phytosporin based on the strain Bacillus subtilis 128 ARRIAM (standard), the third part of seeds was treated with a liquid biopreparation based on the strain Bacillus subtilis 8A (prototype), and the last part of seeds was treated with preparation based on the bacterial strain Bacillus amyloliquefaciens subsp. plantarum BS89 obtained as described above.

Wheat seeds were treated at a rate of 1 l of preparation per 9 l of water with the addition of 20-30 g of sodium carboxymethyl cellulose.

In all trial variants, seeds were sown with an additional application of mineral fertilizers in the form of ammophos (N₆P₅₂) introduced manually under pre-sowing cultivation according to the experimental design. After that, wheat seeds were sown into the sowing zones delimited according to the biopreparation used for treatment as well as the control zone.

Foliage spraying of plants was conducted during the phase of tilling and during the phase of the beginning of booting. Plants were sprayed with the help of a shoulder sprayer at a rate of 1 l of the preparation per 200 l of water per 1 hectare.

The farming culture of wheat corresponded to the zonal technology. The yield was harvested with a SAMPO-130 harvester. It was recalculated to 100% purity and 14% humidity. Gluten content was determined based on GOST 13586.1/68. Statistical assessment of the significance of the obtained results was based on dispersion analysis with a 95% significance level. The results of trials with winter wheat are presented in Table 10.

TABLE 10
The effect of the strain of rhizosphere bacteria on yield and grain
quality of winter wheat, cv. Mironovskaya 808 (Ulyanovsk Region).

| | Yield, | Added yield | | |
|---|---|---|---|---|
| | 100 | 100 | | Gluten |
| Variant | kg/ha | kg/ha | % | % |
| Control | 35.7 | — | — | 20.0 |
| Bacillus subtilis ARRIAM 128 | 39.1 | 3.4 | 9.5 | 22.0 |
| (Phytosporin, standard) | | | | |
| Bacillus subtilis 8A (prototype) | 41.5 | 5.8 | 16.2 | 22.5 |
| Bacillus amyloliquefaciens subsp. | 44.7 | 9.0 | 25.2 | 25.0 |
| plantarum BS89 (claimed) | | | | |

A field trial with spring barley, cv. Preriya, was conducted in Krasnodar Territory on powerful, medium loamy, medium humic chernozem soil, humus after Tyurin 3.24%, nitrate nitrogen 8.3 mg/kg, phosphorus 64 mg/kg, potassium 150 mg/kg, aqueous pH 6.46.

Sowing plot area was 40 m², record plot area 30 m². The trial was conducted in four replications. The land was previously left to lie fallow. The background of fertilizers was N₆P₅₂ (ammophos). Pre-sowing treatment: cultivation at a depth of 5-7 cm. Seeding rate: 4.0 million fertile barley seeds per hectare.

On the day of sowing, a part of barley seeds was left untreated (control), the second part was treated with a liquid biopreparation Phytosporin based on the strain Bacillus subtilis 128 ARRIAM (standard), the third part of seeds was treated with a liquid biopreparation based on the strain Bacillus subtilis 8A (prototype), and the last part of seeds was treated with the preparation based on the bacterial strain Bacillus amyloliquefaciens subsp. plantarum BS89 obtained as described above. Barley seeds were treated at a rate of 1 l of the preparation per 9 l of water with the addition of 20-30 g of sodium carboxymethyl cellulose.

The soil was cultivated before sowing, For this, mineral fertilizers in the form of ammophos (N₆P₅₂) were manually applied according to the experimental scheme. After that, barley seeds were sown into the sowing zones delimited according to the biopreparation used for treatment as well as the control zone.

Foliage spraying was conducted in the phase of 3-5 leaves and in the phase of the end of tillage/beginning of booting. The plants were sprayed with the help of a shoulder sprayer at a rate of 1 l of the preparation per 300 l of water per hectare.

The farming culture of spring barley corresponded to the zonal technology. The yield was harvested with the help of SAMPO-130 harvester and recorded totally, plot-by-plot. It was recalculated to 100% purity and 14% humidity. Protein content was determined according to GOST 10846/91. Statistical assessment of the significance of the results was based on dispersion analysis with a 95% significance level. The results of trials with spring barley are given in Table 11.

TABLE 11
The effect of the strain of rhizosphere bacteria on yield and grain
quality of spring barley, cv. Preriya (Krasnodar Territory).

| | Yield of | Added yield | | Protein content |
|---|---|---|---|---|
| | grain, 100 | 100 | | in grain |
| Strain | kg/ha | kg/ha | % | % |
| Control | 41.0 | — | — | 9.0 |
| Bacillus subtilis ARRIAM 128 | 44.2 | 3.2 | 7.8 | 9.8 |
| (Phytosporin, standard) | | | | |
| Bacillus subtilis 8A (prototype) | 45.0 | 4.0 | 9.9 | 10.2 |
| Bacillus amyloliquefaciens subsp. | 48.3 | 7.3 | 17.8 | 12.5 |
| plantarum BS89 (claimed) | | | | |

Data in Tables 9, 10 and 11 indicate that the microbial preparation based on the claimed bacterial strain Bacillus amyloliquefaciens subsp. plantarum BS89 had a stronger effect on the yield and quality of grain agricultural crops than the biopreparation based on the prototype strain Bacillus subtilis 8A.

A field trial with potato, cv. Nevsky, was conducted in the Leningrad Region on weakly podzol, loamy soil, humus after Tyurin 2.30%, nitrate nitrogen 22.1 mg/kg, phosphorus 315 mg/kg, potassium 52 mg/kg, aqueous pH 5.6.

Sowing material: potato tubers selected from the same preheated and sorted lot. The weight of seed tubers was 60-70 g, eyeholes emerging. Preceding crop: annual grasses for green fodder. Background of fertilizers: organic fertilizers were not introduced under potato, mineral fertilizers were introduced under ridge tillage at a rate of N₁₀₀P₁₁₀K₁₃₀.

Trial plot area was 50 m², record plot area was 25 m². The trial was conducted in four replications. Seeding scheme was 75×30 cm, resulting in the seeding density of 44,000 tubers per hectare. By the time of harvesting, the density of plants made up, on the average, 43,500 plants per hectare.

On the day of sowing, a part of potato tubers was left untreated (control), the second part was treated with a liquid biopreparation Phytosporin based on the strain Bacillus subtilis 128 ARRIAM (standard), the third part of tubers was treated with liquid biopreparation based on the strain Bacillus subtilis 8A (prototype), and the last part of tubers was treated with the preparation based on the bacterial strain Bacillus amyloliquefaciens subsp. plantarum BS89 obtained as described above. Pre-sowing treatment of potato tubers was made at a rate of 1 l of preparation per 9 l of water per 1 ton of potato tubers.

Potato was sown with the help of a clone planter with the planting width of 75 cm and sowing density of 400 tubers per 100 m² into sowing zones delimited according to the biopreparation used for treatment as well as the control zone.

Treatment of plants: treatment between rows—two pre-emergence treatments and one post-emergence treatment; spraying with herbicide Titus (0.03 kg/ha)+Trend (0.2 kg/ha) and insecticide Aktara, water dispersible granules (0.06 kg/ha), boom applicator ON-600 with an application rate of the reference liquid 300 l/ha. Plants in each zone were sprayed with the corresponding preparation with the help of a shoulder sprayer at a rate of 1 l preparation per 300 l of water per hectare: the first spraying in the phase of full sprouts, the second spraying in the phase of budding.

Pre-harvesting removal of herbage: BD-4-7. Harvesting: potato digger KTN-2B with manual tuber picking. The results of trials with potato are presented in Table 12.

TABLE 12
Effectiveness of application of the strain of rhizosphere bacteria
in field experiments with potato, cv. Nevsky (Leningrad Region).

Added yield

| | Yield, | 100 | |
|---|---|---|---|
| Variant | 100 kg/ha | kg/ha | % |
| Control | 191 | — | — |
| Bacillus subtilis ARRIAM 128 | 217 | 26 | 13.6 |
| (Phytosporin, standard) | | | |
| Bacillus subtilis 8A (prototype) | 225 | 34 | 17.8 |
| Bacillus amyloliquefaciens subsp. | 251 | 60 | 31.4 |
| plantarum BS89 (claimed) | | | |

Field microplot trial with white cabbage, cv. Podarok, was conducted in the Leningrad Region on podzolic loamy soil, humus after Tyurin 3.50%, nitrate nitrogen 30.5 mg/kg, phosphorus 280 mg/kg, potassium 40 mg/kg, aqueous pH 6.0.

To obtain sprouts, seeds of white cabbage, cv. Podarok, were sown into boxes with peat-mineral soil. The substrate for the pots was prepared as follows: soddy podzolic sandy loam soil of the trial field of ARRIAM (30%) was mixed with Terravita peat soil (ZAO MNPP Fart) (50%) and washed quartz sand (20%). The solution of azophoska was added to the mixture at a rate of 100% NPK. The substrate obtained in this way was thoroughly mixed, and the boxes were packed with the mixture, 10 kg per box.

For trials with cabbage, 0.1% solution of the following preparations was used: preparation based on strain Bacillus subtilis 128 ARRIAM (Phytosporin-standard); preparation based on the prototype strain Bacillus subtilis 8A; preparation based on the claimed strain Bacillus amyloliquefaciens subsp. plantarum BS89 obtained as described above. A litre of 0.1% solution of one of the biopreparations was applied to each of the three boxes; water was applied to the fourth box.

A nursery garden of cabbage was grown in a greenhouse at 15-20° C. In the phase of 2-3 true leaves, the plants were potted into peat pots with a volume of 0.5 l. As cabbage plants were planted into peat pots, 0.1% solution of the preparation that had been applied to the substrate (water in case of control) was poured to each planting hole, 1 ml of solution (water in case of control) per plant. Sprouts were grown in 30 replications.

After that, cabbage sprouts in the phase of 4-5 true leaves were planted together with a lump of soil in the permanent place in the soil with the planting zones delimited according to the treatment of one of the biopreparations or water. Plot area was 5 m², row spacing 50 cm, spacing of plants in a row 35 cm. The farming culture of cabbage was as is usual. For pest control, cabbage plants were treated twice with a 0.15% solution of the insecticide dimophos (40% emulsion); mineral fertilizers were applied: N₃₅P₂₀K₃₀ three weeks after planting the spouts and N₃₀P₂₀K₃₅ in the phase of leaf rosette.

During vegetation plants in each sowing zone delimited according to the preceding treatment with one of the biopreparations were sprayed twice with a 0.5% solution of the corresponding biopreparation at a rate of 2 i/ha (2 l per 400 ml of water per 1 ha) with the help of a shoulder sprayer SOLO-456. Control plants were not sprayed. The trial was conducted in four replications. The results of trials with cabbage are presented in Table 13.

TABLE 13
Effectiveness of application of the strain of rhizosphere bacteria
in field trials with white cabbage (Leningrad Region).

| | Yield of cabbage, | Added yield | |
|---|---|---|---|
| Experimental variant (bacterial strain) | 100 kg/ha | 100 kg | % |
| Control (without treatment) | 390 | — | — |
| Bacillus subtilis 128 (Phytosporin) | 408 | 18 | 4.6 |
| Bacillus subtilis 8A (prototype) | 415 | 25 | 6.4 |
| Bacillus amyloliquefaciens subsp. | 451 | 61 | 15.6 |
| plantarum BS89 (claimed) | | | |

Data in Tables 12 and 13 indicate that the biopreparation based on the claimed bacterial strain Bacillus amyloliquefaciens subsp. plantarum BS89 has a stronger effect on the yield (productivity) of vegetable agricultural crops than the biopreparation based on the prototype strain Bacillus subtilis 8A.

Field trials on sugar beet, cv. Ramonskaya odnosemennaya 47, were conducted in the educational experimental plot of the Bashkir State Agrarian University (Bashkortostan Republic) on bleached chernozem soil, humus content 5.8%, pH 6.5, phosphorus after Chirikov 91.1 mg/kg, potassium after Chirikov 130.8 mg/kg. Total absorbed bases rather high, 54-56 mg/eq. Degree of bases saturation: 96-98%.

Application rate: ca. 5.5 kg of seeds per hectare. Plant density by the time of harvesting: 80,000 plants/ha. Sowing method broad, row spacing 45 cm, guess row spacing 50 cm. Optimal seeding depth 3-4 cm. Trial plot area 50 m², record plot area 25 m². The trials were conducted in four replications.

The entire sowing area of sugar beet was divided into four zones. In each zone plantings of sugar beet were treated with 0.5% solution of one of the biopreparations: preparation based on strain Bacillus subtilis 128 ARRIAM (Phytosporin-standard); preparation based on the prototype strain Bacillus subtilis 8A; preparation based on the claimed strain Bacillus amyloliquefaciens subsp. plantarum BS89 obtained as described above. Control plants were not treated.

Plants in each sowing zone were sprayed with a 0.5 solution of one of the biopreparations at a rate of 2 l of the biopreparation per 400 ml of water per ha with a shoulder sprayer SOLO-456. Spraying was performed twice: in the phase of 4-6 true leaves and in the phase of closure of herbage in rows. Control plants were not sprayed. The trial was conducted in four replications. The results of trials with sugar beet are presented in Table 14.

TABLE 14
Effectiveness of application the strain of rhizosphere
bacteria in field trials with sugar beet, cv. Ramonskaya
odnosemennaya 47, (Bashkortostan Republic).

| | Yield | | |
|---|---|---|---|
| Experimental variant | sugar beet, | Added yield | |
| (bacterial strain) | 100 kg/ha | 100 kg | % |
| Control (without treatment) | 290 | — | — |
| Bacillus subtilis 128 (Phytosporin) | 322 | 32 | 11.0 |
| Bacillus subtilis 8A (prototype) | 331 | 41 | 14.1 |
| Bacillus amyloliquefaciens subsp. | 355 | 65 | 22.4 |
| plantarum BS89 (claimed) | | | |

Field trials on sunflower and flax were conducted in Stavropol Territory on the soil: medium powerful, medium loamy, medium humic chernozem, humus after Tyurin 3.71%, nitrate nitrogen 6.2 mg/kg, phosphorus 75 mg/kg, potassium 180 mg/kg, aqueous pH 7.15. Farming techniques: preceding crop was spring barley, sunflower and flax were sown with the help of a SKS-610 seed spacing drill with a batch dropper.

Trial variants were: sowing untreated flax and sunflower seeds (control) and sowing seeds treated with biopreparations: Phytosporin (based on the strain Bacillus subtilis 128), the preparation based on the prototype strain Bacillus subtilis 8A and the preparation based on the claimed strain Bacillus amyloliquefaciens subsp. plantarum BS89 at a rate of 2.0 l/t; vegetating plants were also treated with these biopreparations at a rate of 1.0 l/ha.

The trial was microplot, sowing plot area 30 m², record plot area 25 m². The trial was conducted in four replications. Flax and sunflower seeds were harvested with a compact Wintersteiger harvester. The yield was recorded in all trial variants. The results of the experiments are presented in Tables 15 and 16.

TABLE 15
Effectiveness of application of the strain of rhizosphere
bacteria in field trials with flax (Stavropol Territory).

| Experimental variant (preparations | Yield of flax | Added yield | |
|---|---|---|---|
| based on bacterial strain) | seeds, 100 kg/ha | 100 kg | % |
| Control (without treatment) | 7.0 | — | — |
| Bacillus subtilis 128 (Phytosporin) | 8.1 | 1.1 | 15.7 |
| Bacillus subtilis 8A (prototype) | 8.5 | 1.5 | 21.4 |
| Bacillus amyloliquefaciens subsp. | 11.3 | 4.3 | 61.4 |
| plantarum BS89 (claimed) | | | |

TABLE 16
Effectiveness of application of the strain of rhizosphere bacteria
in field trials with sunflower (Stavropol Territory)

| | Yield of | | |
|---|---|---|---|
| Experimental variant | sunflower seeds, | Added yield | |
| (bacterial strain) | 100 kg/ha | 100 kg | % |
| Control (without treatment) | 12.3 | — | — |
| Bacillus subtilis 128 (Phytosporin) | 13.5 | 1.2 | 9.8 |
| Bacillus subtilis 8A (prototype) | 13.7 | 1.4 | 11.4 |
| Bacillus amyloliquefaciens subsp. | 17.1 | 4.8 | 39.0 |
| plantarum BS89 (claimed) | | | |

Data in Tables 14, 15 and 16 indicate that the biopreparation based on the claimed bacterial strain Bacillus amyloliquefaciens subsp. plantarum BS89 has a stronger effect on the yield (productivity) of technical agricultural crops such as sugar beet, flax and sunflower than the biopreparation based on the prototype strain Bacillus subtilis 8A.

The field trials of the biopreparation based on the bacterial strain Bacillus amyloliquefaciens subsp. plantarum BS89 confirmed its high effectiveness for increasing the yield of such agricultural crops as wheat, barley, potato, cabbage, sugar beet, flax and sunflower. These results indicate that this strain is a promising tool for use in agriculture.

In conclusion, the strain of rhizosphere bacteria Bacillus amyloliquefaciens subsp. plantarum BS89 can be used in agriculture as an effective means of increasing plant productivity and protecting plants from diseases caused by phytopathogenic microorganisms. It is a valuable addition to the toolkit of such means.

### REFERENCES

1. Ongena M, Jourdan E, Adam A, Paquot M, Brans A, Joris B, Arpigny J L, Thonart P. 2007. Surfactin and fengycin lipopeptides of Bacillus subtilis as elicitors of induced systemic resistance in plants. Environ Microbiol 9: 1084-1090.
2. Malfanova N, Franzil N, Lugtenberg B, Chebotar V, Ongena M. 2012. Cyclic lipopeptide profile of the plant-beneficial endophytic bacterium Bacillus subtilis C8. Arch Microbiol 194: 893-899.
3. Bais H P, Fall R, Vivanco J M. 2004. Biocontrol of Bacillus subtilis against infection of Arabidopsis roots by Pseudomonas syringae is facilitated by biofilm formation and surfactin production. Plant Physiol 134:307-319.
4. Kloepper J, Leong J, Teintze M. and Schroth M N. 1980. Pseudomonas sideropho-res: a mechanism explaining disease suppressive soils. Current Microbiology 4: 317-320.
5. Chen X.-H., R. Scholz, M. Borriss, H. Junge, G. Mogel, S. Kunz, and R. Borriss. 2009. Difficidin and bacilysin produced by plant-associated Bacillus amyloliquefaciens are efficient in controlling fire blight disease. J. Biotechnol. 140:38-44.
6. Chen X H, Koumoutsi A, Scholz R, Eisenreich A, Schneider K, et al. 2007. Comparative analysis of the complete genome sequence of the plant growth promoting bacterium Bacillus amyloliquefaciens FZB42. Nat Biotechnol 25: 1007-1014.
7. Scholz R, Molohon K J, Nachtigall J, Vater J, Markley A L, Sussmuth R D, Mitchell D A, Borriss R. 2011. Plantazolicin, a novel microcin B17/streptolysin S-like natural product from Bacillus amyloliquefaciens FZB42. J Bacteriol. 193: 215-224.
8. Molohon, K. J.; Melby, J. O.; Lee, J.; Evans, B. S.; Dunbar, K. L.; Bumpus, S. B.; Kelleher, N. L.; Mitchell, D. A. “Structure determination and interception of biosynthetic intermediates for the plantazolicin class of highly discriminating antibiotics.” ACS Chem. Biol., 6:1307-1313 (2011). doi:10.1021/cb200339d
9. He P, Hao K, Blom J, Riickert C, Vater J et al. 2013. Genome sequence of the plant growth promoting strain Bacillus amyloliquefaciens subsp. plantarum B9601-Y2 and expression of mersacidin and other secondary metabolites. J Biotechnol 164: 281-291
10. Palacios O A., Bashan Y, de-Bashan L E. 2014. Proven and potential involvement of vitamins in interactions of plants with plant growth-promoting bacteria: an overview. Biol. Fertil. Soils 50: 415-432.

1. Bacterial strain Bacillus amyloliquefaciens subsp. plantarum BS89 deposited in the collection of the All-Russia Research Institute of Agricultural Microbiology (ARRIAM) under the number RCAM 03458 as a means of increasing the productivity of plants and their protection from diseases caused by phytopathogenic organisms.
